(19) United States
(12) Patent Application Publication
(10) Pub. No.: US 2010/0274042 A1
(43) Pub. Date: Oct. 28, 2010

(54) PROCESS FOR PREPARING (METH)ACRYLIC ESTERS OF ALCOHOLS COMPRISING POLYALKOXY GROUPS

(75) Inventors: Virginie BETTE, Mannheim (DE); Jochen Petzoldt, Weisenheim am Berg (DE); Hermann Bergmann, Singapore (SG)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/764,349

(22) Filed: Apr. 21, 2010

Related U.S. Application Data
(60) Provisional application No. 61/172,237, filed on Apr. 24, 2009.

(30) Foreign Application Priority Data
Apr. 24, 2009 (DE) 10 2009 002 632.0

(51) Int. Cl. C07C 67/02

(52) U.S. Cl. 560/183

(57) ABSTRACT

A process for preparing (meth)acrylic esters (E) of alcohols (A) comprising polyalkoxy groups, in which alcohols (A) which comprise polyalkoxy groups and are of the formula (I)

RO—[(CH₂)ₘO]ₙ—H (I)

in which
m is an integer from 1 to 10,
n is an integer from 1 to 100 and
R is a straight-chain saturated or unsaturated alcohol having 1 to 30 carbon atoms,
   are transesterified with at least one (meth) acrylic ester (D) in the presence of at least one inorganic salt (S).

The present invention relates to a process for catalytically preparing (meth)acrylic esters of alcohols comprising polyalkoxy groups, and to the use thereof.

In the context of the present invention, (“meth)acrylic acid” is understood to mean acrylic acid and/or methacrylic acid, and “(meth)acrylic ester” to mean acrylic ester and/or methacrylic ester. (Meth)acrylic esters are also referred to hereinafter as (meth)acrylates.

(Meth)acrylic esters are usually prepared by catalytic esterification of (meth)acrylic acid or transesterification of other (meth)acrylic esters with alcohols. Frequently, strong acids or bases are used, and so acid- or base-sensitive (meth)acrylic esters generally cannot be prepared in a controlled manner by an esterification or transesterification by this route.

(Meth)acrylic esters of alcohols comprising polyalkoxy groups are known.

EP 0 902 017 A1 discloses (meth)acrylic esters of alcohols comprising polyalkoxy groups. The reaction is effected by means of transesterification with tin catalysts, magnesium alkoxides, lithium, lithium carbonate or lithium hydroxide. From the group of the alcohols comprising polyalkoxy groups, only polyethoxy alcohols with a cetyl/stearyl alkyl chain (C16 alkyl chain) and with a lauryl/myristyl alkyl chain (C14-C12 alkyl chain) are mentioned. According to the examples in this document, a dehydration is required, in the course of which an azeotropic mixture of acrylate and water is distilled off. Owing to this, the catalyst can only be added after removal of the azeotropic mixture.

JP 04 066555 A1 discloses the transesterification of (meth)acrylic esters with C3-C20-alcohols in the presence of tetraalkyl titanate as a catalyst. The alcohols disclosed therein have a short alkyl chain and a short alkoxy chain, for example methoxyethanol and ethoxyethanol.

DE 196 02 035 A1 likewise describes (meth)acrylic esters of alcohols comprising polyalkoxy groups. The transesterification is effected by means of Ca(OH)2, either alone or in combination with LiCl. (Meth)acrylic esters prepared by this method may comprise between 2 and 50 ethylene oxide or propylene oxide units, and a C1-C28-alkyl chain.

EP 0 837 049 A1 discloses a process for synthesizing ethoxylated C₁₀-C₂₀ linear alcohols. The catalysts used are Zr compounds.

It was an object of the present invention to provide a further process with which (meth)acrylic esters of alcohols comprising polyalkoxy groups can be prepared. The synthesis should proceed under mild conditions, so as to result in products with a low color number and high purity. More particularly, the process procedure should be industrially implementable.

The object is achieved by a process for preparing (meth)acrylic esters (E) of alcohols (A) comprising polyalkoxy groups, in which alcohols (A) which comprise polyalkoxy groups and are of the formula (I)

RO—[(CH₂)ₘO]ₙ—H (I)

in which
m is an integer from 1 to 10,
n is an integer from 1 to 100 and
R is a straight-chain saturated or unsaturated alcohol having 1 to 30 carbon atoms,
   are transesterified with at least one (meth) acrylic ester (D) in the presence of at least one inorganic salt (S).

With the aid of the process according to the invention, the preparation of (meth)acrylic esters of alcohols comprising polyalkoxy groups is possible with at least one of the following advantages:
high yield,
mild reaction conditions,
good color numbers and
no washing steps required to purify the reaction mixture.

The alcohols comprising polyalkoxy groups used are typically those in which m is an integer in the range from 1 to 10. m is preferably an integer in the range from 1 to 6, more preferably an integer in the range from 1 to 3. Most preferably, m=2 or 3, such that the unit is an ethylene oxide or propylene oxide unit, especially preferably an ethylene oxide unit.

It will be appreciated that the alcohol comprising polyalkoxy groups used may comprise a plurality of different alkylene oxide units, where the alkylene oxide units may be distributed randomly. The alcohol (A) comprising polyalkoxy groups, however, preferably comprises only one group of alkylene oxide units, preferably ethylene oxide units.

The number n of alkylene oxide units in the alcohol (A) comprising polyalkoxy groups is typically between 1 and 100; in the case that n=1, the alcohol is thus a monoalkoxy alcohol. n is preferably in the range between 5 and 90, more preferably between 10 and 80 and especially preferably between 20 and 50.

The substituent R is a straight-chain saturated or unsaturated alcohol having 1 to 60 carbon atoms. The alcohols may be monoalcohols having 1 to 12 carbon atoms, for example methanol, ethanol, isopropanol, n-propanol, n-butanol, isobutanol, sec-butanol, tert-butanol, n-hexanol, n-heptanol, n-octanol, n-decanol, 2-ethylhexanol.

The substituent R preferably, however, comprises straight-chain saturated or unsaturated primary alcohols having 6 to 22 carbon atoms, known as fatty alcohols. Such fatty alcohols are, for example, hexan-1-ol (hexyl alcohol, caproic alcohol), heptan-1-ol (heptyl alcohol, enanthic alcohol), octan-1-ol (octyl alcohol, capryl alcohol), nonan-1-ol (nonyl alcohol, pelargonyl alcohol), decan-1-ol (decyl alcohol, capric alcohol), undecan-1-ol (undecyl alcohol), undec-10-en-1-ol, dodecan-1-ol (dodecyl alcohol, lauryl alcohol), tridecan-1-ol (tridecyl alcohol), tetradecan-1-ol (tetradecyl alcohol, myristyl alcohol), pentadecan-1-ol (pentadecyl alcohol), hexadecan-1-ol (hexadecyl alcohol, cetyl alcohol, palmityl alcohol), heptadecan-1-ol, heptadecyl alcohol), octadecan-1-ol (octadecyl alcohol, stearyl alcohol), 9-cis-octadecen-1-ol (oleyl alcohol), 9-trans-octadecen-1-ol (elaidyl alcohol), nonadecan-1-ol (nonadecyl alcohol), eicosan-1-ol (eicosyl alcohol, arachyl alcohol), 9-cis-eicosen-1-ol (gadoleyl alcohol), heneicosan-1-ol (heneicosyl alcohol), docosan-1-ol (docosyl alcohol, behenyl alcohol), 13-cis-docosen-1-ol (erucyl alcohol), 13-trans-docosen-1-ol (brassidyl alcohol).

In addition, it is also possible to use higher molecular weight alcohols such as lignoceryl alcohol (C₂₄H₅₀O), ceryl alcohol (C₂₆H₅₄O) or myricyl alcohol (C₃₀H₆₂O) as the substituent R.

Preference is given, however, to using the aforementioned fatty alcohols having 6 to 22 carbon atoms. Preference is given to using fatty alcohols having 8 to 18, preferably having 10 to 16, carbon atoms. The fatty alcohols may also be any desired mixtures of fatty alcohols, for example a mixture of fatty alcohols with a C₁₆- and C₁₈-alkyl chain, with a C₁₃- and C₁₅-alkyl chain, or with a C₁₂- and C₁₄-alkyl chain. In the case of mixtures of fatty alcohols, preference is given to those with a C₁₆- and C₁₈-alkyl chain.

The alcohols which comprise polyalkoxy groups and are usable in the process according to the invention are sold, for example, under the Lutensol® or Pluriol® brand names by BASF SE.

In the reaction step, the transesterification is effected with at least one, preferably exactly one, (meth)acrylic ester (D) in the presence of at least one inorganic salt as a catalyst.

(Meth)acrylic esters (D) are those of a saturated alcohol, preferably saturated C₁-C₁₀-alkyl esters or C₃-C₁₂-cycloalkyl esters of (meth)acrylic acid, more preferably saturated C₁-C₄-alkyl esters of (meth)acrylic acid.

In the context of this document, “saturated” means compounds without C—C multiple bonds (except, of course, the C═C double bond in the (meth)acryloyl units).

Examples of compounds (D) are methyl (meth)acrylate, ethyl (meth)acrylate, n-butyl (meth)acrylate, isobutyl (meth)acrylate, tert-butyl (meth)acrylate, n-octyl (meth)acrylate and 2-ethylhexyl (meth)acrylate, 1,2-ethylene glycol di- and mono(meth)acrylates, 1,4-butanediol di- and mono(meth)acrylates, 1,6-hexanediol di- and mono(meth)acrylates, trimethylolpropane tri(meth)acrylate and pentaerythritol tetra(meth)acrylates.

Particular preference is given to methyl (meth)acrylate, ethyl (meth)acrylate, n-butyl (meth)acrylate and 2-ethylhexyl (meth)acrylate, very particular preference to methyl (meth)acrylate, ethyl (meth)acrylate and n-butyl (meth)acrylate, in particular to methyl (meth)acrylate and ethyl (meth)acrylate and especially to methyl (meth)acrylate.

According to the invention, the catalysts used in the transesterification are inorganic salts. Preference is given to those which have a pK_{B} of not more than 7.0, preferably of not more than 6.0 and more preferably of not more than 4.0. At the same time, the pK_{B} value should not be less than 1.0, preferably not less than 1.5 and more preferably not less than 1.6. Inorganic salts usable in accordance with the invention are preferably heterogeneous inorganic salts.

According to the invention, heterogeneous inorganic salts in the context of this document are those which have a solubility in the reaction medium at 25° C. of not more than 1 g/l, preferably not more than 0.5 g/l and more preferably not more than 0.25 g/l.

The inorganic salt preferably has at least one anion selected from the group consisting of carbonate (CO₃²⁻), hydrogencarbonate (HCO₃⁻), phosphate (PO₄³⁻), hydrogenphosphate (HPO₄²⁻), dihydrogenphosphate (H₂PO₄⁻), sulfate (SO₄²⁻), sulfite (SO₃²⁻) and carboxylate (R¹—COO⁻), in which R¹ is C₁-C₁₈-alkyl, or C₂-C₁₈-alkyl or C₆-C₁₄-aryl optionally interrupted by one or more oxygen and/or sulfur atoms and/or one or more substituted or unsubstituted imino groups.

The collective terms for R¹ specified in the case of carboxylate (R¹—COO—) are each defined as follows:

C₁-C₁₈-alkyl: straight-chain or branched hydrocarbon radicals having up to 18 carbon atoms, preferably C₁-C₁₀-alkyl such as methyl, ethyl, propyl, isopropyl, n-butyl, sec-butyl, tert-butyl, 1,1-dimethylethyl, pentyl, 2-methylbutyl, 1,1-dimethylpropyl, 1,2-dimethylpropyl, 2,2-dimethylpropyl, 1-ethylpropyl, hexyl, 2-methylpentyl, 3-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, 2,4,4-trimethylpentyl, 1,1,3,3-tetramethylbutyl, nonyl and decyl, and isomers thereof.

C₆-C₁₄-aryl: a mono- to tricyclic aromatic ring system comprising 6 to 14 carbon ring members, for example phenyl, naphthyl and anthracenyl, preferably a mono- to bicyclic, more preferably a monocyclic, aromatic ring system.

Preferred anions are phosphate, hydrogenphosphate, sulfate, sulfite and carboxylate, particular preference being given to phosphate.

Phosphate is also understood to mean the condensation products, for example diphosphates, triphosphates and polyphosphates.

The inorganic salt preferably has at least one, more preferably exactly one, cation selected from the group consisting of alkali metals, alkaline earth metals, ammonium, cerium, iron, manganese, chromium, molybdenum, cobalt, nickel and zinc.

Preference is given to alkali metals and particular preference to lithium, sodium or potassium.

Particularly preferred inorganic salts are Li₃PO₄, K₃PO₄, Na₃PO₄, K₂CO₃ and Na₂CO₃ and hydrates thereof, very particular preference being given to K₃PO₄.

According to the invention, K₃PO₄ can be used in anhydrous form, and also as the trihydrate, heptahydrate or nonahydrate.

The inorganic salt can be added in solid form, i.e. in both cases as the pure substance, or dissolved in a suitable solvent. The salt is preferably metered in solid form, in which case no further component which has to be removed in a complicated manner is added to the reaction system.

According to the invention, the inorganic salt used as the catalyst is added completely at the start of the reaction, i.e. not continuously during the course of the reaction. This is advantageous especially with regard to the prior art, since, according to EP 0 902 017 A1, the catalyst can be added only after removal of the water from the reaction mixture. It is therefore advantageous for industrially employable processes, since a staged or continuous addition of the catalyst is frequently impossible owing to technical problems.

The transesterification is effected generally at 30 to 140° C., preferably at 30 to 120° C., more preferably at 40 to 100° C. and most preferably at 60 to 95° C.

In a preferred embodiment of the process according to the invention, the reaction is performed under gentle vacuum of, for example, 200 hPa to standard pressure, preferably 200 to 900 hPa and more preferably 300 to 700 hPa, when the low-boiling alcohol which forms in the transesterification is to be distilled off, optionally as an azeotrope.

The molar ratio between (meth)acrylic ester (D) and alcohol (A) comprising polyalkoxy groups is, in the case of the transesterification catalyzed by an inorganic salt, generally 1-20:1 mol/mol, preferably 1-18:1 mol/mol and more preferably 1-15:1 mol/mol.

The reaction time is generally 45 min to 18 hours, preferably 2 hours to 12 hours and more preferably 3 to 10 hours.

The content of inorganic salts in the reaction medium is generally in the range from about 0.01 to 10 mol %, preferably 0.1-8 and more preferably 0.3-6 mol %, based on the sum of the alcohols (A) comprising polyalkoxy groups used.

In the transesterification, polymerization inhibitors (as described below) are absolutely necessary.

The presence of oxygenous gases (see below) during the performance of the process according to the invention is preferred.

In the inventive transesterification, the products are generally obtained with a color number less than 500 APHA, preferably less than 200 and more preferably less than 150 (to DIN ISO 6271).

The reaction can proceed in organic solvents or mixtures thereof or without addition of solvents. The mixtures are generally substantially anhydrous (i.e. water content less than 10, preferably less than 5, more preferably less than 1 and most preferably less than 0.5% by weight). In addition, the mixtures are substantially free of primary and secondary alcohols, i.e. alcohol content less than 10, preferably less than 5, more preferably less than 1 and most preferably less than 0.5% by weight.

Suitable organic solvents are those known for these purposes, for example tertiary monols such as C₃-C₆-alcohols, preferably tert-butanol, tert-amyl alcohol, pyridine, poly-C₁-C₄-alkylene glycol di-C₁-C₄-alkyl ethers, preferably polyethylene glycol di-C₁-C₄-alkyl ethers, for example 1,2-dimethoxyethane, diethylene glycol dimethyl ether, polyethylene glycol dimethyl ether 500, C₁-C₄-alkylene carbonates, especially propylene carbonate, C₃-C₆-alkyl acetates, especially tert-butyl acetate, THF, toluene, 1,3-dioxolane, acetone, isobutyl methyl ketone, ethyl methyl ketone, 1,4-dioxane, tert-butyl methyl ether, cyclohexane, methylcyclohexane, toluene, hexane, dimethoxymethane, 1,1-dimethoxyethane, acetonitrile, and the mono- or polyphasic mixtures thereof.

In a particularly preferred embodiment of the transesterification, the reaction is performed in the (meth)acrylic ester (D) used as the reactant. Very particular preference is given to performing the reaction in such a way that the product (E), after the reaction has ended, is obtained as an about 10-80% by weight solution in the (meth)acrylic ester (D) used as the reactant, especially as a 20 to 50% by weight solution.

The reactants are present in the reaction medium in dissolved form, suspended as solids, as a melt or in emulsion. As described above, the inorganic salt is preferably used in solid form. The alcohol (A) comprising polyalkoxy groups is preferably used in solid form or as a melt.

The reaction can be effected continuously, for example in a tubular reactor or in a stirred reactor cascade, or batchwise. The inorganic salt is preferably added completely at the start of the reaction, i.e. not continuously during the course of the reaction.

The reaction can be performed in all reactors suitable for such a reaction. Such reactors are known to those skilled in the art. Preference is given to effecting the reaction in a stirred tank reactor or a fixed bed reactor.

For mixing of the reaction mixture, it is possible to use any desired method. Specific stirrer apparatus is not required. The mixing can be effected, for example, by feeding in a gas, preferably an oxygenous gas (see below). The reaction medium may be mono- or polyphasic, and the reactants are dissolved, suspended or emulsified therein. The temperature is adjusted to the desired value during the reaction and can, if desired, be increased or reduced during the course of the reaction.

Alcohols released from the (meth)acrylic esters (D) in the course of transesterification are removed continuously or stepwise in a manner known per se, for example by means of reduced pressure, azeotropic removal, stripping, absorption, pervaporation and diffusion through membranes or extraction.

The stripping can be effected, for example, by passing an oxygenous gas, preferably air or an air-nitrogen mixture, through the reaction mixture, optionally in addition to a distillation.

Suitable methods of absorption are preferably molecular sieves or zeolites (pore size, for example, in the range of about 3-10 angström), or a removal by distillation or with the aid of suitable semipermeable membranes.

However, it is also possible to feed the removed mixture of (meth)acrylic ester (D) and the parent alcohol thereof, which frequently forms an azeotrope, directly into a plant for preparing the (meth)acrylic ester (D), in order to reutilize it there in an esterification with (meth)acrylic acid.

After the reaction has ended, the reaction mixture obtained from the transesterification can be used without further purification, or it can be purified if required in a further step.

In general, in the purification step, the catalyst used is merely removed from the reaction mixture and the reaction product is removed from any organic solvent used.

A removal from the catalyst is generally effected by filtration, electrofiltration, absorption, centrifugation or decantation, or by distillation or rectification. The catalyst removed can subsequently be used for further reactions. In the case of filtration, the reaction mixture can be diluted beforehand in order to achieve a manageable concentration for the removal of the catalyst.

The removal from the organic solvent is effected generally by distillation or rectification, or by filtration in the case of solid reaction products.

In the purification step, however, preference is given to merely removing the catalyst and any solvent used.

The optionally purified reaction mixture can be subjected to a distillation in which the (meth)acrylic ester (E) of the alcohols comprising polyalkoxy groups is separated by a distillation from unconverted (meth)acrylic ester (D) and any by-products formed.

The distillation units are usually rectification columns of customary design with a circulation evaporator and condenser. The feed is preferably effected into the bottom region; the bottom temperature here is, for example, 130-160° C., preferably 150-160° C., the top temperature is preferably 140-145° C. and the top pressure is 3-20, preferably 3 to 5, mbar. It will be appreciated that the person skilled in the art can also determine other temperature and pressure ranges in which the particular (meth)acrylic ester (E) of the alcohols comprising polyalkoxy groups can be purified by distillation. What is essential here is a separation of the desired product from reactants and by-products under conditions under which the desired product is subject to a minimum degree of degradation reaction.

The distillation unit has generally 5 to 50 theoretical plates.

The distillation units are of a design known per se and have the customary internals. Useful column internals include in principle all common internals, for example trays, structured packagings and/or random packings. Among the trays, preference is given to bubble-cap trays, sieve trays, valve trays, Thormann trays and/or dual-flow trays; among the random packings, preference is given to those comprising rings, helices, saddles, Raschig, Intos or Pall rings, barrel or Intalox saddles, Top-Pak, etc., or braids.

The desired product is preferably distilled batchwise, which first removes low boilers from the reaction mixture, usually solvents or unconverted (meth)acrylic esters (D). After removal of these low boilers, the distillation temperature is increased and/or the vacuum is reduced, and the desired product is distilled off.

The remaining distillation residue is usually discarded.

In a preferred embodiment of the process according to the invention, the catalyst is first removed as described above; this removal is preferably effected by filtration. In this case, it may be necessary that the reaction mixture is diluted with a suitable solvent before filtration in order to achieve a manageable concentration for the removal of the catalyst. Thereafter, any solvent present is removed by distillation and replaced by another solvent.

Alternatively, any solvent present can first be removed by distillation and optionally replaced by another solvent, without any need to remove the catalyst from the reaction mixture. The latter can subsequently be removed by the aforementioned methods.

In both preferred embodiments described, after distillative removal of the solvent, the latter is replaced by another suitable solvent. Possible solvents for use include, for example, other (meth)acrylic esters, (meth)acrylic acid, alcohols, customary organic solvents, water and any desired mixtures thereof. The new solvent used is preferably (meth)acrylic acid, more preferably methacrylic acid.

After purification, the end product (E) may comprise very small amounts of reactants, especially of the original (meth)acrylic ester (D). The proportion of (meth)acrylic ester (D) in the (meth)acrylic ester (E) is typically less than 20% by weight, preferably less than 10% by weight and more preferably less than 5% by weight.

The reaction conditions in the inventive transesterification are mild. Owing to the low temperatures and other mild conditions, the formation of by-products in the reaction is prevented, which can otherwise originate, for example, from strongly acidic or basic catalysts, or result from undesired free-radical polymerization of the (meth)acrylic esters (D) used, which can otherwise be prevented only by addition of stabilizers.

In the inventive reaction regime, additional stabilizer can be added to the reaction mixture over and above the storage stabilizer present in the (meth)acrylic ester (D) in any case, for example hydroquinone monomethyl ether, phenothiazine, phenols, for example 2-tert-butyl-4-methylphenol, 6-tert-butyl-2,4-dimethylphenol, or N-oxyls such as 4-hydroxy-2,2,6,6-tetramethylpiperidine N-oxyl, 4-oxo-2,2,6,6-tetramethylpiperidine N-oxyl or Uvinul® 4040P from BASF SE, or amines such as Kerobit® BPD from BASF SE (N,N′-di-sec-butyl-p-phenylenediamine), for example in amounts of 50 to 2000 ppm.

Advantageously, the transesterification is performed in the presence of an oxygenous gas, preferably air or air-nitrogen mixtures.

The (meth)acrylic esters (E), prepared in accordance with the invention, of alcohols comprising polyalkoxy groups find use, for example, as monomers or comonomers in the preparation of dispersions, for example acrylic dispersions, as reactive diluents, for example in radiation-curable coating materials or in paints, preferably in exterior paints, and in dispersions for use in the paper sector, in the cosmetics sector, in the pharmaceutical sector, in agrochemical formulations, in the textile industry and in the oil extraction sector.

The examples which follow are intended to illustrate the properties of the invention, but without restricting it.

### EXAMPLES

Unless stated otherwise, “parts” in this document are understood to mean “parts by weight”.

### Example 1

### Transesterification of Methyl Methacrylate with Pulverulent Lutensol®AT25 in the Presence of K₃PO₄

The transesterification was effected in a 750 ml Miniplant reactor with Oldershaw column and liquid distributor. The return ratio was 25:1 (return:output), the stirrer speed (anchor stirrer) was 300 rpm and the air introduction rate was 1.5 l/h.

This apparatus was initially charged with 75 mg of hydroquinone monomethyl ether (120 ppm), 143 g (1.43 mol) of methyl methacrylate (MMA), 479.2 g (0.35 mol) of a pulverulent polyethoxy alcohol (Lutensol®AT25 from BASF SE, degree of ethoxylation approx. 25, M_{w} approx. 1360) and 1.51 g (2 mol %) of potassium phosphate, which were stirred. The mixture was subsequently warmed stepwise to 75° C. and the vacuum was set (300 mbar). During the reaction, distillate (MMA and methanol) was removed continuously and partly recycled (return ratio 25:1). The temperature in the bottom was between 75° C. and 95° C.; the vapor temperature was between 46° C. and 52° C. After 4 h, the reaction was ended and the vacuum was broken. The suspension was cooled.

The unpurified bottom product (200.66 g) was subsequently analyzed by means of NMR and OH number; it comprised 50 mol % of MMA and 50 mol % of polyethoxy methacrylate.

Subsequently, the crude product was diluted to a 25% by weight solution in MMA and filtered. This gave a clear colorless solution (25% by weight in MMA, APHA color number 42) of the polyethoxy methacrylate end product. The number of OH groups was determined; it was less than 1 mg KOH/g. The potassium content was less than 0.001 g/100 g. The turbidity value in methanol (1:10) was 0.16 NTU.

### Example 2

### Transesterification of Methyl Methacrylate with Molten Lutensol®AT25 in the Presence of K₃PO₄

The transesterification was effected in a 750 ml Miniplant reactor with Oldershaw column and liquid distributor. The return ratio was 25:1 (return:output), the stirrer speed (anchor stirrer) was 300 rpm and the air introduction rate was 1.5 l/h.

This apparatus was initially charged with 75 mg of hydroquinone monomethyl ether (120 ppm) and 143 g (1.43 mol) of methyl methacrylate (MMA), which were stirred. Subsequently, the bath temperature was adjusted to 75° C. As the bottom temperature reached 48° C., 479.2 g (0.35 mol) of a polyethoxy alcohol (Lutensol®AT25 from BASF SE, degree of ethoxylation approx. 25, M_{w} approx. 1360), which had been melted beforehand, and 1.13 g (1.5 mol %) of potassium phosphate were added thereto, and the vacuum was set (300 mbar). During the reaction, distillate (MMA and methanol) was removed continuously and partly recycled (return ratio 25:1). The temperature in the bottom was between 70° C. and 95° C.; the vapor temperature was between 46° C. and 52° C. After 4 h, the reaction was ended and the vacuum was broken. The suspension was cooled.

The unpurified bottom product (569.96 g) was subsequently analyzed by means of NMR; it comprised 20 mol % of MMA and 80 mol % of polyethoxy methacrylate.

The crude product was purified as described in example 1.

### Comparative Examples 1 to 4

### Transesterification of Methyl Methacrylate with Molten Lutensol®AT25 in the Presence of Other Catalysts

Example 2 was repeated, except that the catalysts specified in table 1 were used. In all noninventive reactions (referred to as comparative examples in the table), there was polymerization and premature stoppage of the reaction. The results of the reaction are likewise summarized in table 1.

| | Catalyst | |
|---|---|---|
| Example | (2 mol %) | Result |
| Example 2 | K₃PO₄¹⁾ | after 4 h: 80 mol % of polyethoxy |
| | | methacrylate in MMA |
| Comparative | DBTO²⁾ | stoppage after 90 min owing to |
| Example 1 | | polymerization |
| Comparative | LiOH (98%) | stoppage after 65 min owing to |
| Example 2 | | polymerization |
| Comparative | Ti(OiPr)₄ | stoppage after 240 min owing to |
| Example 3 | | polymerization |
| Comparative | K₂CO₃ | stoppage after 180 min owing to |
| Example 4 | | polymerization |
| Comparative | KOH | stoppage after 210 min, viscous bottom |
| Example 5 | | comprises, according to NMR, only reactant |
| | | (52 mol %) and MMA (48 mol %). |

¹⁾1.5 mol % of K₃PO₄;
²⁾dibutyltin oxide

### Example 3

### Transesterification of Methyl Methacrylate with Pulverulent Lutensol®AT25 in the Presence of K₃PO₄

The transesterification was effected in a 4 l Miniplant reactor with a column filled with Sulzer stainless steel random packing, and a liquid distributor. The return ratio was 25:1 (return:output), the stirrer speed (propeller stirrer) was 300 rpm and the air introduction rate was 1.5 l/h.

This apparatus was initially charged with 240 mg of hydroquinone monomethyl ether (120 ppm), 1000 g (10 mol) of methyl methacrylate (MMA), 1000 g (0.74 mol) of a pulverulent polyethoxy alcohol (Lutensol®AT25 from BASF SE, degree of ethoxylation approx. 25, M_{w}, approx. 1360) and 9.42 g (6 mol %) of potassium phosphate, which were stirred. Subsequently, the mixture was heated stepwise to 85° C. and the vacuum was set (580 mbar). During the reaction, distillate (MMA and methanol) was removed continuously and partly recycled (return ratio 25:1). The temperature in the bottom was between 87° C. and 93° C.; the vapor temperature was between 82° C. and 83° C. The kinetics of the reaction were monitored by means of NMR, which indicated complete conversion after only 4.5 h. After 7 h, the reaction was ended and the vacuum was broken. The suspension was cooled.

The unpurified bottom product (1714 g) was subsequently analyzed by means of NMR; it comprised 88 mol % of MMA and 12 mol % of polyethoxy methacrylate.

Subsequently, the crude product was diluted to a 25% by weight solution in MMA and filtered. This gave a clear colorless solution (25% by weight in MMA) of the polyethoxy methacrylate end product.

### Example 4

### Transesterification of Methyl Methacrylate with Lutensol® AT25 and Subsequent Solvent Replacement

The transesterification was effected in a 4 l Miniplant reactor with a column filled with Sulzer stainless steel random packing, and a liquid distributor. The return ratio was 25:1 (return:output), the stirrer speed (propeller stirrer) was 300 rpm and the air introduction rate was 1.5 l/h.

This apparatus was initially charged with 240 mg of hydroquinone monomethyl ether (120 ppm), 1000 g (10 mol) of methyl methacrylate (MMA), 1000 g (0.74 mol) of a pulverulent polyethoxy alcohol (Lutensol®AT25 from BASF SE, degree of ethoxylation approx. 25, M_{w} approx. 1360) and 9.42 g (6 mol %) of potassium phosphate, which were stirred. Subsequently, the mixture was heated stepwise to 85° C. and the vacuum was set (590-600 mbar). During the reaction, distillate (MMA and methanol) was removed continuously and partly recycled (return ratio 25:1). The temperature in the bottom was between 87° C. and 93° C.; the vapor temperature was between 82° C. and 83° C. After 6 h, the vacuum was reduced to 100 mbar, then to 6 mbar. The temperature in the bottom was kept at 50 to 60° C. At a temperature of 60° C., 1045 g (12 mol) of methacrylic acid were added to the suspension.

The unpurified bottom product (2100 g) was subsequently analyzed by means of NMR (49% by weight of methacrylic acid, 2% by weight of MMA, 45% by weight of polyethoxy methacrylate, 2% by weight of polyethoxy alcohol); it comprised 9.4 g of residual K₃PO₄ catalyst.

### Example 5

### Transesterification of Methyl Methacrylate with Lutensol®A7N

The transesterification was effected in a 4 l Miniplant reactor with a column filled with Sulzer stainless steel random packing, and a liquid distributor. The return ratio was 25:1 (return:output), the stirrer speed (propeller stirrer) was 300 rpm and the air introduction rate was 1.5 l/h.

This apparatus was initially charged with 240 mg of hydroquinone monomethyl ether (120 ppm), 1000 g (10 mol) of methyl methacrylate (MMA), 1000 g (2 mol) of a pulverulent polyethoxy alcohol (Lutensol®A7N from BASF SE, degree of ethoxylation approx. 7, M_{w} approx. 508) and 16.98 g (4 mol %) of potassium phosphate, which were stirred. Subsequently, the mixture was heated stepwise to 70° C. and the vacuum was set (300 mbar). During the reaction, distillate (MMA and methanol) was removed continuously and partly recycled (return ratio 25:1). The temperature in the bottom was between 70° C. and 77° C.; the vapor temperature was between 50° C. and 65° C. After 7 h, the reaction was ended and the vacuum was broken. The suspension was cooled and filtered through a fluted filter.

This gave a viscous solution (1525 g, APHA color number 89), which was then analyzed by means of NMR. The suspension comprised 63 mol % of MMA and 37 mol % of polyethoxy methacrylate.

### Example 6

### Transesterification of Methyl Methacrylate with Pluriol® A1000E

The transesterification was effected in a 750 ml Miniplant reactor with an Oldershaw column and liquid distributor. The return ratio was 25:1 (return:output), the stirrer speed (anchor stirrer) was 300 rpm and the air introduction rate was 1.5 l/h.

This apparatus was initially charged with 102 mg of hydroquinone monomethyl ether (120 ppm), 500 g (5 mol) of methyl methacrylate (MMA), 500 g (0.5 mol) of a pulverulent polyethoxy alcohol (Pluriol® A1000E from BASF SE, degree of ethoxylation approx. 22, M_{w} approx. 1000) and 4.25 g (4 mol %) of potassium phosphate, which were stirred. Subsequently, the mixture was heated stepwise to 75° C. and the vacuum was set (400 mbar). During the reaction, distillate (MMA and methanol) was removed continuously and partly recycled (return ratio 25:1). The temperature in the bottom was between 77° C. and 82° C.; the vapor temperature was between 46° C. and 74° C. After 5.5 h, the reaction was ended and the vacuum was broken. The suspension was cooled and filtered through a fluted filter. Subsequently, excess MMA was evaporated off.

This gave a white solid product (486 g), which was then analyzed by means of NMR. The product comprised >98 mol % of polyethoxy methacrylate and <1% by weight of polyethoxy alcohol. The number of OH groups was determined; it was 0.3 mg KOH/g.

### Example 7

The transesterification was effected in a 750 ml Miniplant reactor with an Oldershaw column and liquid distributor. The return ratio was 25:1 (return:output), the stirrer speed (anchor stirrer) was 300 rpm and the air introduction rate was 1.5 l/h.

This apparatus was initially charged with 65.5 mg of hydroquinone monomethyl ether (120 ppm), 300 g (3 mol) of methyl methacrylate (MMA), 246 g (0.25 mol) of a pulverulent polyethoxy alcohol (MeO—(CH₂CH(Me)O)₉—(CH₂CH₂O)₁₀—H, OH number=57, M_{w} approx. 948) and 2.13 g (4 mol %) of potassium phosphate, which were stirred. Subsequently, the mixture was heated stepwise to 70° C. and the vacuum was set (400 mbar). During the reaction, distillate (MMA and methanol) was removed continuously and partly recycled (return ratio 25:1). The temperature in the bottom was between 70° C. and 77° C.; the vapor temperature was between 50° C. and 65° C. After 3 h, the reaction was ended and the vacuum was broken.

The suspension was cooled and filtered through a fluted filter. Subsequently, excess MMA was evaporated off.

This gave a viscous solution (240 g), which was then analyzed by means of NMR. It comprised 2 mol % of polyethoxy alcohol and 95 mol % of polyethoxy methacrylate.

1. A process for preparing (meth)acrylic esters (E) of alcohols (A) comprising polyalkoxy groups, in which alcohols (A) which comprise polyalkoxy groups and are of the formula (I)
RO—[(CH₂)ₘO]ₙ—H (I)
in which
m is an integer from 1 to 10,
n is an integer from 1 to 100 and
R is a straight-chain saturated or unsaturated alcohol having 1 to 30 carbon atoms,
are transesterified with at least one (meth) acrylic ester (D) in the presence of at least one inorganic salt (S).

2. The process according to claim 1, wherein m in the formula (I) is an integer in the range from 1 to 3.

3. The process according to claim 2, wherein the alkoxy group is an ethylene oxide or propylene oxide unit.

4. The process according to any of the preceding claims, wherein n in the formula (I) is an integer in the range from 10 to 80.

5. The process according to claim 4, wherein n in the formula (I) is an integer in the range from 20 to 50.

6. The process according to any of the preceding claims, wherein the substituent R is a straight-chain saturated or unsaturated primary alcohol having 6 to 22 carbon atoms.

7. The process according to any of the preceding claims, wherein the inorganic salt (K1) has at least one anion which is selected from the group consisting of carbonate (CO₃²⁻), hydrogencarbonate (HCO₃⁻), phosphate (PO₄³⁻), hydrogenphosphate (HPO₄²⁻), dihydrogenphosphate (H₂PO₄⁻), sulfate (SO₄²⁻), sulfite (SO₃²⁻) and carboxylate (R¹—COO⁻), in which R¹ is C₁-C₁₈-alkyl, or C₂-C₁₈-alkyl or C₆-C₁₄-aryl optionally interrupted by one or more oxygen and/or sulfur atoms and/or one or more substituted or unsubstituted imino groups.

8. The process according to any of the preceding claims, wherein the inorganic salt has at least one cation which is selected from the group consisting of alkali metals, alkaline earth metals, ammonium, cerium, iron, manganese, chromium, molybdenum, cobalt, nickel and zinc.

9. The process according to any of the preceding claims, wherein the inorganic salt is selected from the group consisting of Li₃PO₄, K₃PO₄, Na₃PO₄, K₂CO₃ and Na₂CO₃ and hydrates thereof.

10. The process according to any of the preceding claims, wherein the (meth)acrylic ester (D) is a saturated C₁-C₁₀-alkyl ester.

11. The process according to any of the preceding claims, wherein the (meth)acrylic ester (D) is selected from the group consisting of methyl (meth)acrylate, ethyl (meth)acrylate, n-butyl (meth)acrylate and 2-ethylhexyl (meth)acrylate.

12. The use of a (meth)acrylic ester (E) prepared by a process according to any of the preceding claims as a monomer or comonomer in the preparation of dispersions, for example acrylic dispersions, as a reactive diluent, such as in radiation-curable coating materials or in paints, and in dispersions for use in the paper sector, in the cosmetics sector, in the pharmaceutical sector, in agrochemical formulations, in the textile industry and in the oil extraction sector.
